(19) United States
(12) Patent Application Publication
(10) Pub. No.: US 2024/0428940 A1
(43) Pub. Date: Dec. 26, 2024

(54) INFORMATION PROCESSING APPARATUS, INFORMATION PROCESSING METHOD, AND COMPUTER-READABLE RECORDING MEDIUM

(71) Applicant: DEEPEYEVISION INC., Shimotsuke-shi, Tochigi (JP)

(72) Inventor: Yusuke KONDO, Shimotsuke-shi, Tochigi (JP)

(21) Appl. No.: 18/699,072

(22) PCT Filed: Oct. 20, 2022
(86) PCT No.: PCT/JP2022/039094

§ 371 Date: Apr. 5, 2024

(30) Foreign Application Priority Data
Oct. 20, 2021 (JP) 2021-171543

(51) Int. Cl. G16H 50/20; G06V 10/764; G16H 30/20; G16H 30/40

(52) U.S. Cl. CPC G16H 50/20; G06V 10/764; G16H 30/20; G16H 30/40

(57) ABSTRACT

To provide an information processing apparatus, an information processing method, and a computer-readable recording medium that can present areas highly likely to be abnormal areas in images without the need for learning data to which annotation information specifying abnormal areas in medical images on a pixel by pixel basis has been given.

An image acquisition unit 224 that acquires a first image acquired by photographing an examination region of a patient, an image classification unit 226 that acquires a level of certainty that the examination region in the first image is healthy and/or a level of certainty that the examination region is unhealthy, an image creation unit 227 that creates a second image by visualizing an area contributing to classification by the image classification unit, and an output unit 228 that outputs inference results based on the level of certainty acquired by the image classification unit and the second image created by the image creation unit are included.

### CROSS-REFERENCE TO RELATED APPLICATION

The present application is based on Japanese Patent Application No. 2021-171543 filed on Oct. 20, 2021, the contents of which are incorporated herein by reference.

### TECHNICAL FIELD

The present disclosure relates to an information processing apparatus, an information processing method, and a computer-readable recording medium.

### BACKGROUND ART

In the medical field, a specialist, a radiologist, or the like performs annotation, i.e., specifies an abnormal area in a medical image by drawing a contour, causes a machine learning model to do learning based on the medical image to which annotation information has been given, and extracts a feature value of the abnormal area using a learned model acquired by learning.

### CITATION LIST

### Patent Literature

Patent Literature 1: International Publication No. WO 2019/142910

### SUMMARY OF INVENTION

Patent Literature 1 discloses a diagnostic support apparatus that identifies an area with abnormal blood circulation in a fundus image using a learned model that has learned a relationship between fundus images, which are images of the fundus, and areas with abnormal blood circulation in the fundus images based on the fundus images and the areas with abnormal blood circulation identified based on fluorescein fundus angiographic images.

To generate a highly accurate learned model, large quantities of medical images to which accurate annotation information has been given are necessary. However, to obtain accurate annotation information, it is necessary to secure one or more radiologists with high expertise as well as sufficient working hours, involving a great deal of cost.

When annotation information is given for a diffuse disease in which lesions spread in a wide area, it is necessary to point out abnormal areas, borders of which are not sharply-defined, and in pointing out abnormal areas, there can be discrepancies even among radiologists with high expertise.

Thus, an object of the present disclosure is to provide an information processing apparatus, an information processing method, and a computer-readable recording medium that can present areas highly likely to be abnormal areas in images without the need for learning data to which annotation information specifying abnormal areas in medical images on a pixel by pixel basis has been given.

An information processing apparatus according to one aspect of the present disclosure comprises: an image acquisition unit that acquires a first image acquired by photographing an examination region of a patient; an image classification unit that acquires a level of certainty that the examination region in the first image is healthy and/or a level of certainty that the examination region is unhealthy; an image creation unit that creates a second image by visualizing an area contributing to classification by the image classification unit; and an output unit that outputs inference results based on the level of certainty acquired by the image classification unit and the second image created by the image creation unit.

The present aspect can present the area highly likely to be an abnormal area in an image by visualizing the area contributing greatly to the level of certainty that the examination region is unhealthy based on the level of certainty acquired by the image classification unit. Furthermore, the present aspect can grasp the area highly likely to be an abnormal area in the image without generating a learned model caused to do learning using a large volume of learning data given annotation information that specifies abnormal areas on a pixel by pixel basis.

In the information processing apparatus, the image classification unit acquires the level of certainty that the examination region is healthy and/or the level of certainty that the examination region is unhealthy by inputting the first image to an inference model, and the inference model may be a model that estimate whether the examination region included in the first image is healthy or unhealthy. The present aspect makes it possible to easily acquire a level of certainty having desired accuracy.

In the information processing apparatus, based on the level of certainty that the examination region is unhealthy, the image creation unit may create the second image by adjusting a value representing contribution to the classification by the image classification unit. The present aspect makes it possible to accurately point out an abnormal area on an image including an area predicted to be unhealthy.

A method according to another aspect of the present disclosure comprises: acquiring a first image acquired by photographing an examination region of a patient; acquiring a level of certainty that the examination region in the first image is healthy and/or a level of certainty that the examination region in the first image is unhealthy; creating a second image in which an area contributing to the level of certainty that the examination region is unhealthy is visualized; and outputting inference results based on the acquired level(s) of certainty and the second image.

A computer-readable recording medium according to another aspect of the present disclosure records a program that causes one or more computers to perform the processes of: acquiring a first image acquired by photographing an examination region of a patient; acquiring a level of certainty that the examination region in the first image is healthy and/or a level of certainty that the examination region in the first image is unhealthy; creating a second image in which an area contributing to the level of certainty that the examination region is unhealthy is visualized; and outputting inference results based on the acquired level(s) of certainty and the second image.

An information processing apparatus according to another aspect of the present disclosure comprises: a learning unit that input an image labeled with healthiness or unhealthiness as ground truth data to a machine learning model and cause the machine learning model to do learning; and a model output unit that outputs a learned model caused to do learning by the learning unit.

The present aspect makes it possible to obtain a learned model that can be used to infer a diagnosis predicted from images. Note that at the time of learning, since images labeled with either only “healthy” or “unhealthy” on an image by image basis can be used as learning data, the learning data can be collected more easily than conventional learning data that needs annotation information specifying an abnormal area on a pixel by pixel basis.

In the information processing apparatus, the image may include a plurality of images labeled, as ground truth data, with either healthiness or unhealthiness regarding a plurality of diseases including diffuse diseases. The present aspect makes it possible to obtain a learned model that has learned features of unhealthiness, spreading over a plurality of diseases including, in particular, diffuse diseases, of which it can be difficult to point out abnormal areas.

A method according to another aspect of the present disclosure comprises: acquiring a plurality of images labeled with either healthiness or unhealthiness as ground truth data; causing a machine learning model to do learning using the images, where the machine learning model estimates whether an examination region included in the images is healthy or unhealthy; and outputting a learned model obtained through learning done by the machine learning model.

### Advantageous Effects of Invention

The present disclosure can provide an information processing apparatus, an information processing method, and a computer-readable recording medium that can present areas highly likely to be abnormal areas in images without the need for learning data to which annotation information specifying abnormal areas in medical images on a pixel by pixel basis has been given.

### BRIEF DESCRIPTION OF DRAWINGS

FIG. 1 is a diagram showing a network configuration of an information processing system according to one embodiment.

FIG. 2 is a schematic diagram showing processes of a learning apparatus according to one embodiment.

FIG. 3 is a schematic diagram showing processes of an inference apparatus according to one embodiment.

FIG. 4 is a block diagram of the learning apparatus according to one embodiment.

FIG. 5 is a block diagram of the inference apparatus according to one embodiment.

FIG. 6 is a flowchart showing a learning process of the learning apparatus according to one embodiment.

FIG. 7 is a flowchart showing an inference process of the inference apparatus according to one embodiment.

FIG. 8 is a diagram showing a function used to adjust density of a heat map according to one embodiment.

FIG. 9 is a schematic diagram showing processes of a learning apparatus according to one embodiment.

FIG. 10 is a flowchart showing an inference process of an inference apparatus according to one embodiment.

### DESCRIPTION OF EMBODIMENTS

Embodiments of the present invention will be described with reference to the accompanying drawings. Note that the embodiments described below are intended to facilitate the understanding of the present invention, but are not to be interpreted as limiting the present invention. Also, various changes can be made to the present invention without departing from the gist of the present invention. Furthermore, those skilled in the art can adopt embodiments obtained by replacing elements described below with equivalents and such embodiments are also included in the scope of the present invention.

### (System Configuration)

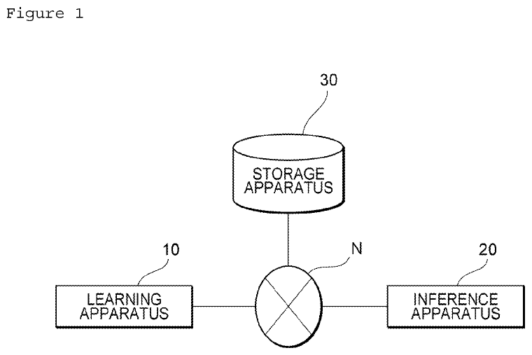

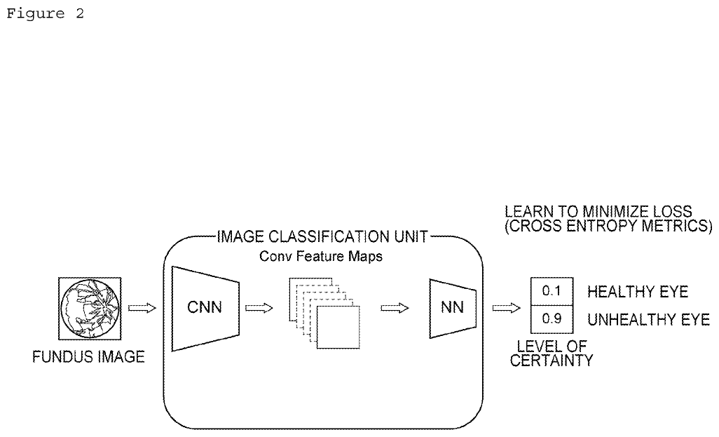

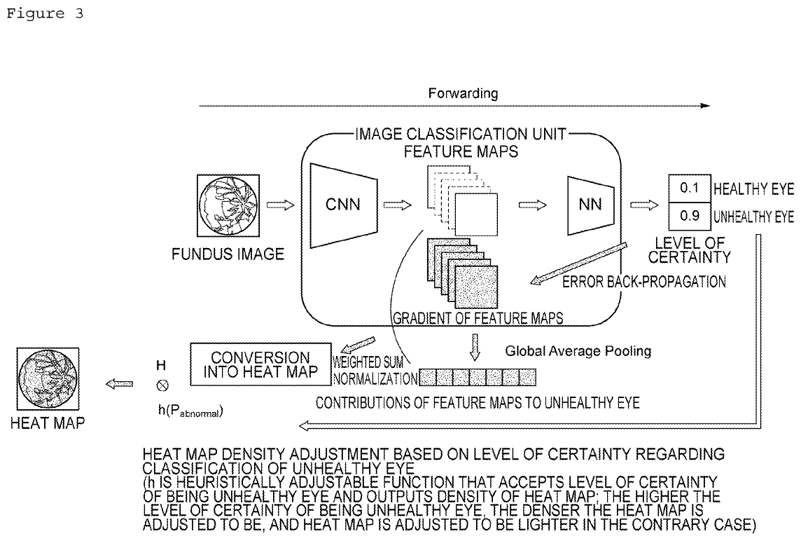

The present disclosure will be outlined with reference to FIGS. 1, 2, and 3. FIG. 1 is a diagram showing a network configuration of an information processing system according to one embodiment. FIG. 2 is a schematic diagram showing processes of a learning apparatus according to one embodiment. FIG. 3 is a schematic diagram showing processes of an inference apparatus according to one embodiment.

The information processing system includes a learning apparatus 10, an inference apparatus 20, and a storage apparatus 30. The learning apparatus 10 is connected to the inference apparatus 20 and the storage apparatus 30 via a communications network N. The communications network N may be either a wired communications network or a wireless communications network made up of a wired or wireless circuit, or the Internet or a local area network (LAN).

The learning apparatus 10 causes machine learning models to do learning based on learning data stored in the storage apparatus 30 and stores learned models in the storage apparatus 30. Although the learning apparatus 10 according to the present embodiment includes the machine learning models, the machine learning models may be provided separately from the learning apparatus 10.

Here, the machine learning model has a certain model structure and process parameters that change with a learning process and has its identification accuracy improved when the process parameters are optimized based on experience obtained from learning data. That is, the machine learning model learns optimum process parameters through a learning process. Regarding algorithms for the machine learning model, for example, Support Vector Machine, Logistic Regression, and Neural Network are available for use, but the type of neural network is not specifically limited. Some of the machine learning models that undergo the learning have not undergone any learning yet, and the others have already undergone some learning using learning data.

Note that the learned model is a model that has done learning in advance using appropriate learning data in contrast to the machine learning model that does learning based on any machine learning algorithm. However, it is not that the learned model no longer does any more learning and the learned model can do additional learning.

The inference apparatus 20 outputs output data corresponding to characteristics of input data using a learned model. The inference apparatus 20 according to the present embodiment makes inferences using a learned model acquired from the storage apparatus 30. Here, acquiring a learned model means acquiring information needed to reproduce functions of the learned model on the inference apparatus 20. For example, when a neural network is used as a machine learning model, acquiring a learned model means acquiring at least information about the number of layers in the neural network, the number of nodes in each layer, weight parameters of links interconnecting the nodes, bias parameters of the respective nodes, and function forms of activation functions of the respective nodes.

The storage apparatus 30 stores learning data used for learning done by the machine learning model. The storage apparatus 30 according to the present embodiment stores, as learning data, fundus images labeled with either “healthy” or “unhealthy” as ground truth data. The storage apparatus 30 also stores learned models outputted by the learning apparatus 10. Although shown as a single storage apparatus in FIG. 1, the storage apparatus 30 may be made up of one or more file servers. Although in the present embodiment, a fundus image labeled with either “healthy” or “unhealthy” is used as an example of learning data, according to another embodiment, medical images acquired by photographing another examination region of a patient and labeled with either “healthy” or “unhealthy” may be used as learning data.

The learning apparatus 10 according to the present embodiment includes a machine learning model that accepts fundus images as input data and classifies the fundus images into images of healthy eyes and images of unhealthy eyes as shown in FIG. 2. The learning apparatus 10 classifies the fundus images using the machine learning model and causes a machine learning model to do learning so as to minimize differences between predicted results and ground truth data labeled to learning data. Although in the present embodiment, description is given of a case in which fundus images, which are an example of medical images, are used, according to another embodiment, images of the kidneys, images of the liver, or similar other images acquired by photographing another examination region of a patient may be used.

Since fundus images labeled with either only “healthy” or “unhealthy” on an image by image basis can be used as learning data, the learning data can be collected more easily than conventional learning data that needs annotation information specifying an abnormal area on a pixel by pixel basis.

As shown in FIG. 3, the inference apparatus 20 according to the present embodiment conducts forward calculations using a learned model, and infers whether a fundus image belongs to a healthy eye or an unhealthy eye. Next, the inference apparatus 20 performs error back-propagation from an output layer corresponding to the unhealthy eye to a convolutional layer desired to be visualized, calculates contributions of feature maps to output of the unhealthy eye, weights feature maps obtained by the forward calculations with contributions, sums the weighted feature maps, and thereby creates a heat map.

By visualizing an area contributing greatly to the output of an unhealthy eye using a learned model that has learned features of unhealthiness from large quantities of images of healthy eyes and images of unhealthy eyes, it is possible to present an abnormal area in an unhealthy eye which is difficult to clearly define over a plurality of diseases. Furthermore, the inference apparatus 20 according to the present embodiment can grasp the area highly likely to be an abnormal area in the fundus image without generating a learned model caused to do learning using a large volume of learning data given annotation information that specifies abnormal areas on a pixel by pixel basis. Although in the present embodiment, abnormal areas in fundus images, which are an example of medical images, are presented, according to another embodiment, abnormal areas in medical images such as images of the kidneys or images of the liver acquired by photographing another examination region of a patient may be presented.

### (Functional Configuration: Learning Apparatus)

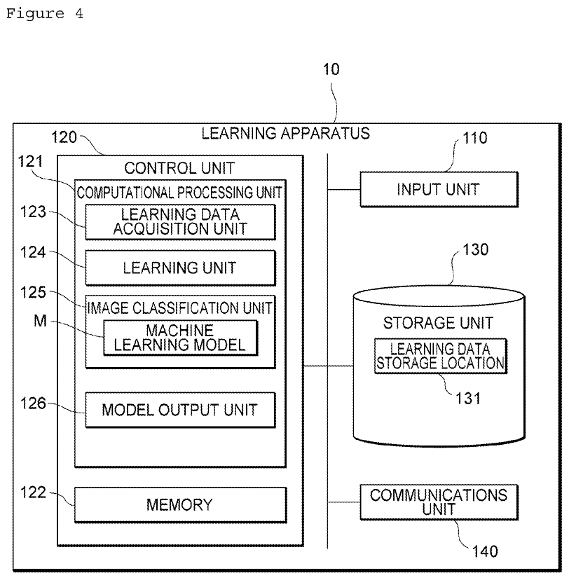

FIG. 4 is a block diagram of the learning apparatus according to one embodiment. Note that only necessary functional components are shown in FIG. 4 by assuming a single learning apparatus 10, but the learning apparatus 10 may be configured as part of a multi-functional distributed system made up of multiple computer systems.

The learning apparatus 10 includes an input unit 110, a control unit 120, a storage unit 130, and a communications unit 140.

The input unit 110, which is configured to accept operations from an administrator of the learning apparatus 10, can be implemented by a keyboard, a mouse, a touch panel, and the like.

The control unit 120 includes a computational processing unit 121, such as a CPU or an MPU, which corresponds to a processor, and a memory 122 such as a RAM. Based on various types of input, the computational processing unit 121 (processor) executes a program stored in the storage unit 130 by loading the program into the memory 122 and thereby implements after-mentioned functions and processes of the computational processing unit 121. The program may be installed on a computer by being stored in a non-transitory computer-readable recording medium such as a CD-ROM or by being distributed through a network. The memory 122 functions as working memory needed by the computational processing unit 121 (processor) in order to execute the program.

The storage unit 130, which is made up of a storage apparatus such as a hard disk, records various programs needed by the control unit 120 in order to perform processes as well as data and the like necessary for execution of the various programs. In the present embodiment, desirably the storage unit 130 includes a learning data storage location 131.

The learning data storage location 131 stores learning data used for learning done by a machine learning model M described later. According to the present embodiment, the learning data storage location 131 stores fundus images labeled with either “healthy” or “unhealthy” as ground truth data. Note that although according to the present embodiment, it is assumed that the learning data storage location 131 stores a plurality of fundus images labeled, as ground truth data, with either “healthy” or “unhealthy” regarding a plurality of diseases including diffuse diseases, according to other embodiments, the learning data storage location 131 may store a plurality of fundus images labeled, as ground truth data, with either “healthy” or “unhealthy” regarding a single diffuse disease or a plurality of fundus images labeled, as ground truth data, with either “healthy” or “unhealthy” regarding a plurality of diseases without including diffuse diseases.

The communications unit 140 is configured to connect the learning apparatus 10 to a network. For example, the communications unit 140 can be implemented by a LAN card, an analog modem, an ISDN modem, or the like as well as an interface for use to connect these devices to a processing unit via a transmission path such as a system bus.

Furthermore, as shown in FIG. 4, the computational processing unit 121 includes a learning data acquisition unit 123, a learning unit 124, an image classification unit 125, and a model output unit 126 as functional components.

The learning data acquisition unit 123 acquires learning data used for learning done by a machine learning model M described later and stores the learning data in the learning data storage location 131.

According to the present embodiment, the learning data acquisition unit 123 acquires fundus images labeled with either “healthy” or “unhealthy” as ground truth data from the storage apparatus 30 and stores the fundus images in the learning data storage location 131.

Using the learning data acquired by the learning data acquisition unit 123, the learning unit 124 causes the machine learning model M to do learning. According to the present embodiment, the learning unit 124 inputs fundus images labeled with either “healthy” or “unhealthy” as ground truth data to the image classification unit 125 described later and causes the machine learning model M to do learning.

The image classification unit 125 accepts fundus images as input and classifies the fundus images into images of healthy eyes and images of unhealthy eyes. According to the present embodiment, the image classification unit 125 accepts fundus images as input, and outputs levels of certainty of being a healthy eye and an unhealthy eye using the machine learning model M.

The machine learning model M accepts fundus images as input data and classifies the fundus images into images of healthy eyes and images of unhealthy eyes. In the present embodiment, as an example of the machine learning model M, description will be given of the use of a convolutional neural network (CNN) that accepts fundus images as input data and outputs classifications of the images. However, the CNN is only an example of the machine learning model M, and the learning apparatus 10 may use another component as the machine learning model M.

The learning unit 124 causes the machine learning model M to do learning so as to minimize differences between results predicted by the machine learning model M and ground truth data labeled to learning data.

When the machine learning model M finishes learning, the model output unit 126 outputs a learned model obtained as a result of the learning done by the machine learning model M to the storage apparatus 30. Note that the learning unit 124 may finish learning, for example, after making the machine learning model M learn a certain number of learning data or when accuracy of classification predicted using the machine learning model M satisfies certain conditions.

### (Functional Configuration: Inference Apparatus)

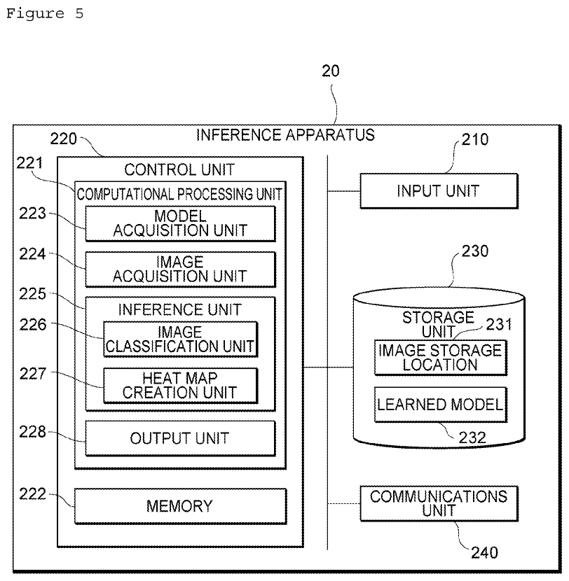

FIG. 5 is a block diagram of the inference apparatus according to one embodiment. Note that only necessary functional components are shown in FIG. 5 by assuming a single inference apparatus 20, but the inference apparatus 20 may be configured as part of a multi-functional distributed system made up of multiple computer systems.

The inference apparatus 20 includes an input unit 210, a control unit 220, a storage unit 230, and a communications unit 240.

The input unit 210, which is configured to accept operations from an administrator of the inference apparatus 20, can be implemented by a keyboard, a mouse, a touch panel, and the like.

The control unit 220 includes a computational processing unit 221, such as a CPU or an MPU, which corresponds to a processor, and a memory 222 such as a RAM. Based on various types of input, the computational processing unit 221 (processor) executes a program stored in the storage unit 230 by loading the program into the memory 222 and thereby implements after-mentioned functions and processes of the computational processing unit 221. The program may be installed on a computer by being stored in a non-transitory computer-readable recording medium such as a CD-ROM or by being distributed through a network. The memory 222 functions as working memory needed by the computational processing unit 221 (processor) in order to execute the program.

The storage unit 230, which is made up of a storage apparatus such as a hard disk, records various programs needed by the control unit 220 in order to perform processes as well as data and the like necessary for execution of the various programs. According to the present embodiment, desirably the storage unit 230 includes an image storage location 231, and a learned model 232.

The image storage location 231 stores images to be used for inference. According to the present embodiment, the image storage location 231 stores fundus images from which a diagnosis is inferred.

The learned model 232 stores learned models used for inference. According to the present embodiment, the learned model 232 stores learned models that accept fundus images as input data and classify the fundus images into images of healthy eyes and images of unhealthy eyes. In the present embodiment, a description will be given of an example that uses, as an example of the learned model 232, a convolutional neural network (CNN) that accepts a fundus image as input data, and classifies the fundus images into images of healthy eyes and images of unhealthy eyes. However, the CNN is only an example of the learned model 232, and the inference apparatus 20 may use a learned model database of another configuration as the learned model 232.

The communications unit 240 is configured to connect the inference apparatus 20 to a network. For example, the communications unit 240 can be implemented by a LAN card, an analog modem, an ISDN modem, or the like as well as an interface for use to connect these devices to a processing unit via a transmission path such as a system bus.

Furthermore, as shown in FIG. 5, the computational processing unit 221 includes, as functional components, a model acquisition unit 223, an image acquisition unit 224, an inference unit 225, an image classification unit 226, a heat map creation unit 227, and an output unit 228.

The model acquisition unit 223 acquires learned models used for inference and stores the learned models in the learned model 232. According to the present embodiment, the model acquisition unit 223 acquires learned models from the storage apparatus 30 and stores the learned models in the learned model 232.

The image acquisition unit 224 acquires the images to be used for inference. According to the present embodiment, the image acquisition unit 224 acquires the fundus images for use to infer a diagnosis, from the image storage location 231.

The inference unit 225 infers a diagnosis predicted from images acquired by the image acquisition unit 224. According to the present embodiment, the inference unit 225 is made up of the image classification unit 226 and the heat map creation unit 227. First, the inference unit 225 inputs fundus images to the image classification unit 226 and acquires levels of certainty of being a healthy eye and an unhealthy eye from the image classification unit 226.

The image classification unit 226 accepts fundus images as input and classifies the fundus images into images of healthy eyes and images of unhealthy eyes. According to the present embodiment, as shown in FIG. 3, the image classification unit 226 accepts fundus images as input, conducts forward calculations using a learned model 232, and outputs levels of certainty of being a healthy eye and an unhealthy eye.

The heat map creation unit 227 creates a heat map, visualizing areas that have contributed to classification by the image classification unit. According to the present embodiment, as shown in FIG. 3, when levels of certainty of being a healthy eye and an unhealthy eye are outputted, the heat map creation unit 227 performs error back-propagation from an output layer corresponding to the unhealthy eye to a convolutional layer desired to be visualized, calculates a gradient of feature maps with respect to output of the unhealthy eye to calculate contributions of respective feature maps to the output of the unhealthy eye, and finds global max pooling (GMP) of the gradient. Next, the heat map creation unit 227 weights the feature maps obtained by the forward calculations with the GMP, and acquires a coefficient map by adding up all the feature maps.

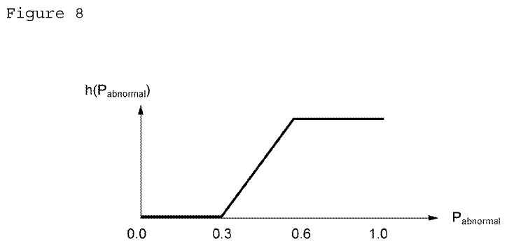

According to one embodiment, the heat map creation unit 227 adjusts the value of each element of the coefficient map based on the level of certainty of being an unhealthy eye. For example, as shown in FIG. 8, when the level of certainty of being an unhealthy eye is 0.0 to 0.3, the heat map creation unit 227 may set the value of each element of the coefficient map to zero; when the level of certainty of being an unhealthy eye is 0.3 to 0.6, the heat map creation unit 227 may adjust the value of each element in proportion to the level of certainty of being an unhealthy eye; and when the level of certainty of being an unhealthy eye is 0.6 to 1.0, the heat map creation unit 227 may make adjustments to set the value of each element to 100%, i.e., to keep the value as it is. By decreasing the value of each element of the coefficient map when the level of certainty of being an unhealthy eye is low and maintaining the value of each element of the coefficient map when the level of certainty is high, it is possible to accurately point out an abnormal area on an image predicted to be unhealthy.

Finally, the heat map creation unit 227 creates a heat map by converting the coefficient map into an image using a color scale and resizing the resulting image to the size of an input image.

Whereas an example of creating a heat map using Grad-CAM (Gradient-weighted Class Activation Mapping) is described in the present embodiment, a heat map may be acquired using another visualization technique. Besides, whereas a heat map that represents the magnitude of contribution with a color scale is created in the present embodiment, a visualized map that represents the magnitude of contribution in another form may be created.

The output unit 228 outputs inference results that are based on information acquired by the inference unit 225. According to the present embodiment, the output unit 228 outputs the fundus image acquired by the image acquisition unit 224 by superimposing the heat map on the fundus image using alpha blending.

### (Learning Process)

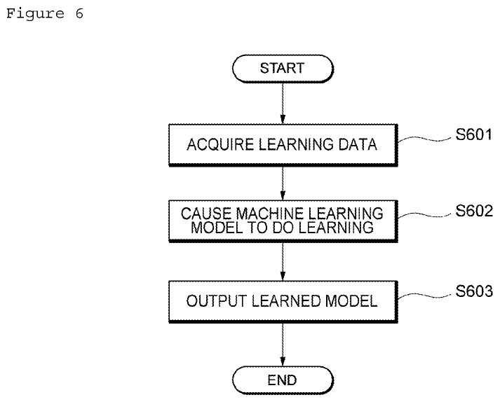

A learning process of the learning apparatus according to one embodiment will be described in detail with reference to FIG. 6. According to the present embodiment, it is assumed that before the learning process described in FIG. 6 is performed, learning data is stored in the storage apparatus 30 under the supervision of the administrator of the learning apparatus 10. Note that the process shown in FIG. 6 is performed, for example, when the administrator enters a command via the input unit 110 to perform the process of generating a learned model.

In step S601, the learning data acquisition unit 123 of the learning apparatus 10 acquires learning data used for learning done by a machine learning model M and stores the learning data in the learning data storage location 131. According to the present embodiment, the learning data acquisition unit 123 acquires fundus images labeled with either “healthy” or “unhealthy” as ground truth data from the storage apparatus 30 and stores the fundus images in the learning data storage location 131. Note that according to the present embodiment, it is assumed that the learning data acquisition unit 123 acquires a plurality of fundus images labeled, as ground truth data, with either “healthy” or “unhealthy” regarding a plurality of diseases including diffuse diseases and stores the fundus images in the learning data storage location 131. That is, if one or more of the plurality of diseases including diffuse diseases are relevant, the fundus images are labeled with “unhealthy” as ground truth data.

Next, in step S602, using the learning data acquired by the learning data acquisition unit 123, the learning unit 124 of the learning apparatus 10 causes the machine learning model M to do learning. According to the present embodiment, the learning unit 124 inputs fundus images labeled with either “healthy” or “unhealthy” as ground truth data to the image classification unit 125 of the learning apparatus 10 and causes the machine learning model M to do learning.

The machine learning model M accepts fundus images as input data and classifies the fundus images into images of healthy eyes and images of unhealthy eyes. In the present embodiment, as an example of the machine learning model M, description will be given of the use of a convolutional neural network (CNN) that accepts fundus images as input data and outputs classifications of the images. However, the CNN is only an example of the machine learning model M, and the learning apparatus 10 may use another component as the machine learning model M.

The learning unit 124 causes the machine learning model M to do learning so as to minimize differences between results predicted by the machine learning model M and ground truth data labeled to learning data.

When the machine learning model M finishes learning, in step S603, the model output unit 126 of the learning apparatus 10 outputs a learned model obtained as a result of the learning done by the machine learning model M to the storage apparatus 30. Note that the learning unit 124 may finish learning, for example, after making the machine learning model M learn a certain number of learning data or when accuracy of classification predicted using the machine learning model M satisfies certain conditions.

### (Inference Process)

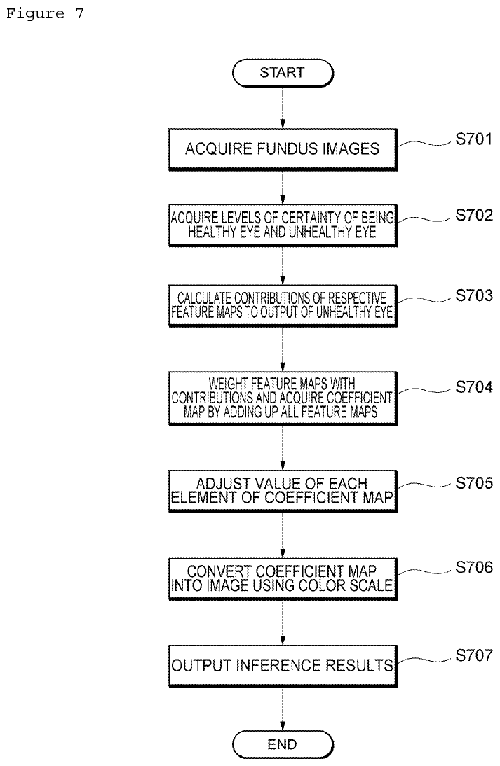

An inference process of the inference apparatus according to one embodiment will be described in detail with reference to FIG. 7. According to the present embodiment, it is assumed that before the inference process described in FIG. 7 is performed, a learned model acquired from the storage apparatus 30 is stored as the learned model 232 under the supervision of the administrator of the inference apparatus 20. It is also assumed that a fundus image to be used for inference is stored in the image storage location 231 of the inference apparatus 20. Note that the process shown in FIG. 7 is performed, for example, when the administrator enters a command via the input unit 210 to perform the inference process.

In step S701, the image acquisition unit 224 of the inference apparatus 20 acquires the images to be used for inference. According to the present embodiment, the image acquisition unit 224 acquires the fundus images for use to infer a diagnosis, from the image storage location 231.

In step S702, the inference unit 225 of the inference apparatus 20 acquires levels of certainty of being a healthy eye and an unhealthy eye. According to the present embodiment, the inference unit 225 inputs fundus images to the image classification unit 226 of the inference apparatus 20 and acquires the levels of certainty of being a healthy eye and an unhealthy eye from the image classification unit 226. Specifically, the image classification unit 226 accepts fundus images as input, conducts forward calculations using a learned model 232, and outputs the levels of certainty of being a healthy eye and an unhealthy eye.

In the present embodiment, a convolutional neural network (CNN) that accepts fundus images as input data and classifies the fundus images into images of healthy eyes and images of unhealthy eyes is used as an example of the learned model 232. However, the CNN is only an example of the learned model 232, and the inference apparatus 20 may use another component as the learned model 232.

Once the levels of certainty of being a healthy eye and an unhealthy eye are outputted, in step S703, the heat map creation unit 227 of the inference apparatus 20 performs error back-propagation from an output layer corresponding to the unhealthy eye to a convolutional layer desired to be visualized, calculates a gradient of feature maps with respect to output of the unhealthy eye to calculate contributions of respective feature maps to the output of the unhealthy eye, and finds global max pooling (GMP) of the gradient.

In step S704, the heat map creation unit 227 weights the feature maps obtained by the forward calculations with the GMP, and acquires a coefficient map by adding up all the feature maps. Then, in step S705, the heat map creation unit 227 adjusts the value of each element of the coefficient map based on the level of certainty of being an unhealthy eye.

According to the present embodiment, as shown in FIG. 8, when the level of certainty of being an unhealthy eye is 0.0 to 0.3, the heat map creation unit 227 sets the value of each element of the coefficient map to zero; when the level of certainty of being an unhealthy eye is 0.3 to 0.6, the heat map creation unit 227 adjusts the value of each element in proportion to the level of certainty of being an unhealthy eye; and when the level of certainty of being an unhealthy eye is 0.6 to 1.0, the heat map creation unit 227 makes adjustments to set the value of each element to 100%, i.e., to keep the value as it is. By decreasing the value of each element of the coefficient map when the level of certainty of being an unhealthy eye is low and maintaining the value of each element of the coefficient map when the level of certainty is high, it is possible to accurately point out an abnormal area on an image predicted to be unhealthy eye.

Furthermore, in step S706, the heat map creation unit 227 creates a heat map by converting the adjusted coefficient map into an image using a color scale and resizing the resulting image to the size of an input image.

Whereas in the present embodiment, description has been given of an example in which a heat map is created using Grad-CAM (Gradient-weighted Class Activation Mapping), a heat map may be acquired using another visualization technique. Besides, whereas a heat map that represents the magnitude of contribution with a color scale is created in the present embodiment, a visualized map that represents the magnitude of contribution in another form may be created.

In step S707, the output unit 228 of the inference apparatus 20 outputs inference results that are based on information acquired by the inference unit 225. According to the present embodiment, the output unit 228 outputs the fundus image acquired by the image acquisition unit 224 by superimposing the heat map on the fundus image using alpha blending.

Thus, according to the present embodiment, the learning apparatus 10 can obtain a learned model that can be used to infer a diagnosis predicted from fundus images. Note that in learning, since the fundus images labeled with either only “healthy” or “unhealthy” on an image by image basis can be used as learning data, the learning data can be collected more easily than conventional learning data that needs annotation information specifying an abnormal area on a pixel by pixel basis.

According to the present embodiment, by visualizing an area contributing greatly to the output of an unhealthy eye using a learned model that has learned features of unhealthiness from large quantities of images of healthy eyes and images of unhealthy eyes, the inference apparatus 20 can present an abnormal area in an unhealthy eye which is difficult to clearly define over a plurality of diseases. Furthermore, the inference apparatus 20 according to the present embodiment can grasp the area highly likely to be an abnormal area in the fundus image without generating a learned model caused to do learning using a large volume of learning data given annotation information that specifies abnormal areas on a pixel by pixel basis.

Although in the present embodiment, abnormal areas in fundus images, which are an example of medical images, are presented, according to another embodiment, abnormal areas in medical images such as images of the kidneys or images of the liver acquired by photographing another examination region of a patient may be presented.

### Other Embodiments

So far, description has been given of an example in which an inference process is performed using a single learned model obtained by causing a single machine learning model to do learning using learning data made up of images labeled, as ground truth data, with healthiness or unhealthiness regarding a plurality of diseases. In the following embodiment, description will be given of an example in which an inference process is performed using a plurality of learned models M-1 to M-N obtained by causing each of machine learning models to do learning regarding respective one of a plurality of diseases using learning data made up of images labeled with healthiness or unhealthiness as ground truth data.

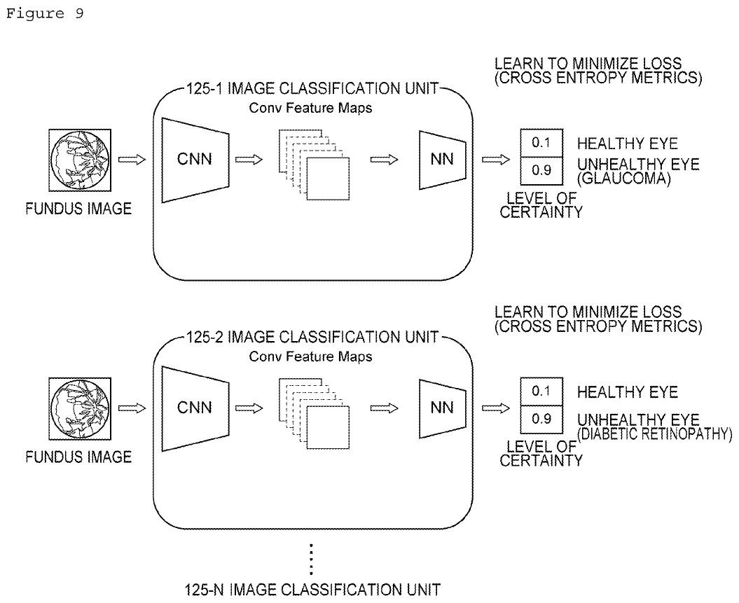

The learning apparatus 10 according to the present embodiment, includes a plurality of machine learning models that accept fundus images as input data and classifies the fundus images into images of healthy eyes and images of unhealthy eyes (disease) as shown in FIG. 9. The learning apparatus 10 classifies the fundus images using the machine learning models and causes each of the machine learning models to do learning so as to minimize differences between predicted results and ground truth data labeled to learning data. For example, an image classification unit 125-1 is a machine learning model that learn features of glaucoma, and classifies images into images of healthy eyes and images of unhealthy eyes (glaucoma). On the other hand, an image classification unit 125-2 is a machine learning model that learn features of diabetic retinopathy, and classifies images into images of healthy eyes and images of unhealthy eyes (diabetic retinopathy).

The learning data storage location 131 stores respective sets of learning data used for learning done by a plurality of machine learning models M-1 to M-N. According to the present embodiment, the learning data storage location 131 stores fundus images used for learning done by the machine learning model M-1 by being labeled with either “healthy” or “unhealthy (glaucoma)” as ground truth data, fundus images used for learning done by the machine learning model M-2 by being labeled with either “healthy” or “unhealthy (diabetic retinopathy)” as ground truth data, and fundus images used for learning done by the machine learning model M-N by being labeled with either “healthy” or “unhealthy (disease N)” as ground truth data.

The inference apparatus 20 according to the present embodiment conducts forward calculations using a plurality of learned models, and infers whether a fundus image belongs to a healthy eye or an unhealthy eye. The inference apparatus 20 includes a plurality of learned models 232-1 to 232-N that have learned features of a plurality of diseases, respectively, and acquires the level of certainty of being a healthy eye and the level of certainty of being an unhealthy eye (disease) on a disease by disease basis. According to the present embodiment, the inference unit 225 includes a plurality of image classification units 226-1 to 226-N and a plurality of heat map creation units 227-1 to 227-N corresponding to the respective ones of the plurality of image classification units. First, the inference unit 225 inputs fundus images to the plurality of image classification units 226-1 to 226-N and acquires the levels of certainty of being a healthy eye and an unhealthy eye (disease) from each of the image classification units 226.

Next, the inference unit 225 selects one or more image classification units 226 from the plurality of image classification units 226-1 to 226-N based on the levels of certainty obtained from the plurality of image classification units 226-1 to 226-N, and instructs the corresponding heat map creation unit(s) 227 to create a heat map visualizing areas that have contributed to the classification by the selected image classification unit(s) 226. According to the present embodiment, the inference unit 225 selects one image classification unit 226 having the highest level of certainty concerning an unhealthy eye (disease) among the levels of certainty of being a healthy eye and an unhealthy eye (disease) acquired from the image classification units 226 and causes the heat map creation unit 227 corresponding to the selected image classification unit 226 to create a heat map.

Whereas in the present embodiment, the inference unit 225 selects one of the image classification units 226, according to other embodiments, the inference unit 225 may select one or more image classification units 226 with a level of certainty of being an unhealthy eye (disease) equal to or higher than a certain threshold such as 0.5 or a certain number of image classification units 226 with top levels of certainty concerning an unhealthy eye (disease).

### (Learning Process)

Regarding a learning process, each of the plurality of machine learning models M-1 to M-N can be caused to learn according to procedures similar to those used for learning of a single machine learning model described in FIG. 6, and thus description thereof will be omitted.

### (Inference Process)

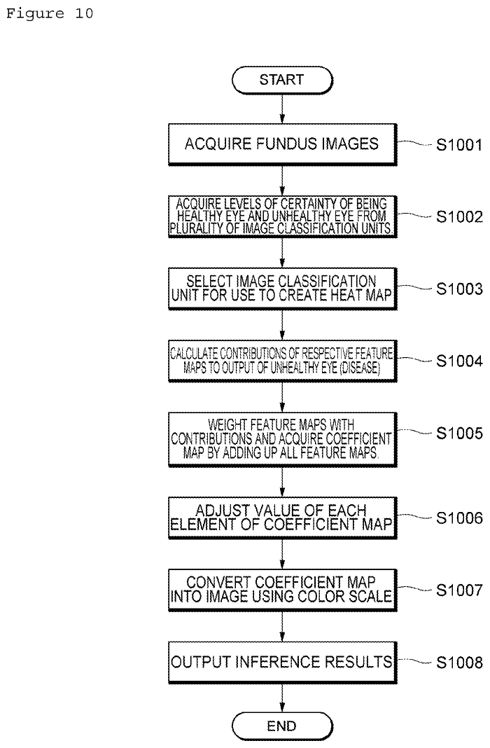

An inference process of an inference apparatus according to one embodiment will be described in detail with reference to FIG. 10. According to the present embodiment, it is assumed that before the inference process described in FIG. 10 is performed, a plurality of learned models 232-1 to 232-N acquired from the storage apparatus 30 are stored as the learned models 232 under the management of an administrator of the inference apparatus 20. It is also assumed that fundus images to be used for inference are stored in the image storage location 231 of the inference apparatus 20. Note that the process shown in FIG. 10 is performed, for example, when the administrator enters a command via the input unit 210 to perform the inference process.

In step S1001, the image acquisition unit 224 acquires the images to be used for inference. According to the present embodiment, the image acquisition unit 224 acquires the fundus images for use to infer a diagnosis, from the image storage location 231.

In step S1002, the inference unit 225 inputs fundus images to the plurality of image classification units 226-1 to 226-N and acquires the levels of certainty of being a healthy eye and an unhealthy eye (disease) from each of the image classification units.

Once the levels of certainty of being a healthy eye and an unhealthy eye (disease) are outputted, in step S1003, the inference unit 225 selects one or more image classification units 226 from the plurality of image classification units 226-1 to 226-N based on the levels of certainty obtained from the plurality of image classification units 226-1 to 226-N. According to the present embodiment, the inference unit 225 selects one image classification unit 226 having the highest level of certainty concerning an unhealthy eye (disease) among the levels of certainty of being a healthy eye and an unhealthy eye (disease) acquired from the image classification units 226.

In step S1004, the heat map creation unit 227 performs error back-propagation from an output layer corresponding to the unhealthy eye (disease) of the selected image classification unit 226 to a convolutional layer desired to be visualized, calculates a gradient of feature maps with respect to output of the unhealthy eye (disease) to calculate contributions of respective feature maps to the output of the unhealthy eye (disease), and finds global max pooling (GMP) of the gradient.

In step S1005, the heat map creation unit 227 weights the feature maps obtained by the forward calculations with the GMP, and acquires a coefficient map by adding up all the feature maps. Then, in step S1006, the heat map creation unit 227 adjusts the value of each element of the coefficient map based on the level of certainty of being an unhealthy eye (disease).

According to the present embodiment, as shown in FIG. 8, when the level of certainty of being an unhealthy eye (disease) is 0.0 to 0.3, the heat map creation unit 227 sets the value of each element of the coefficient map to zero; when the level of certainty of being an unhealthy eye (disease) is 0.3 to 0.6, the heat map creation unit 227 adjusts the value of each element in proportion to the level of certainty of being an unhealthy eye (disease); and when the level of certainty of being an unhealthy eye (disease) is 0.6 to 1.0, the heat map creation unit 227 makes adjustments to set the value of each element to 100%, i.e., to keep the value as it is.

Furthermore, in step S1007, the heat map creation unit 227 creates a heat map by converting the adjusted coefficient map into an image using a color scale and resizing the resulting image to the size of an input image.

In step S1008, the output unit 228 outputs inference results that are based on information acquired by the inference unit 225. According to the present embodiment, the output unit 228 outputs the fundus image acquired by the image acquisition unit 224 by superimposing the heat map on the fundus image using alpha blending.

Thus, according to the present embodiment, the learning apparatus 10 can obtain a plurality of learned models each of which has learned features of a specific disease and can be used to infer diagnosis predicted from fundus images. Note that in learning, since the fundus images labeled with either only “healthy” or “unhealthy” on an image by image basis can be used as learning data, the learning data can be collected more easily than conventional learning data that needs annotation information specifying an abnormal area on a pixel by pixel basis.

According to the present embodiment, by using the plurality of learned models each of which has learned features of a specific disease and by visualizing an area contributing greatly to the output of the unhealthy eye (disease) to which the relevant image classification unit has given a high level of certainty, the inference apparatus 20 can present an abnormal area in the unhealthy eye which is difficult to define clearly. Furthermore, the inference apparatus 20 according to the present embodiment can grasp the area highly likely to be an abnormal area in the fundus image without generating a learned model caused to do learning using a large volume of learning data given annotation information that specifies abnormal areas on a pixel by pixel basis.

An information processing apparatus according to one aspect of the present disclosure may comprise a plurality of image classification units, and select one or more image classification units based on levels of certainty acquired from the plurality of image classification units, and the image creation unit may create one or more second images by visualizing areas contributing to the classification by the selected image classification unit(s). According to the present aspect, using levels of certainty concerning features of a plurality of different types of unhealthiness, the information processing apparatus can present the area highly likely to be such an abnormal area in the fundus image that has originated in the features of the selected type of unhealthiness.

In the information processing apparatus, each of the plurality of image classification units acquires a level of certainty that an examination region is healthy and/or a level of certainty that the examination region has a specific disease by inputting a first image to an inference model, and the inference model for each of the image classification units may infer whether the examination region included in the first image is healthy or has a specific disease. According to the present aspect, the information processing apparatus can easily acquire a level of certainty with a desired accuracy regarding each of a plurality of different diseases.

### REFERENCE SIGNS LIST

10 . . . learning apparatus, 110 . . . input unit, 120 . . . control unit, 121 . . . computational processing unit, 122 . . . memory, 123 . . . learning data acquisition unit, 124 . . . learning unit, 125 . . . image classification unit, 126 . . . model output unit, 130 . . . storage unit, 131 . . . learning data storage location, 140 . . . communications unit, 20 . . . inference apparatus, 210 . . . input unit, 220 . . . control unit, 221 . . . computational processing unit, 222 . . . memory, 223 . . . model acquisition unit, 224 . . . image acquisition unit, 225 . . . inference unit, 226 . . . image classification unit, 227 . . . heat map creation unit (image creation unit), 228 . . . output unit, 230 . . . storage unit, 231 . . . image storage location, 232 . . . learned model, 240 . . . communications unit, 30 . . . storage apparatus, M . . . machine learning model, N . . . communications network

1. An information processing apparatus for presenting an area highly likely to be a specific area in a first image concerning an examination region of an examinee, the apparatus information processing comprising:
an image acquisition unit that acquires the first image;
an image classification unit that acquires a level of certainty that the examination region in the first image is healthy and/or a level of certainty that the examination region is unhealthy by inputting the first image to an inference model, wherein the inference model is a model obtained by using a plurality of images labeled with either healthiness or unhealthiness as ground truth data on an image by image basis;
an image creation unit that creates a second image by visualizing an area contributing to classification by the image classification unit; and
an output unit that outputs inference results based on the level of certainty acquired by the image classification unit and the second image created by the image creation unit.

2. The information processing apparatus according to claim 1,
wherein the information processing apparatus comprises a plurality of image classification units, and selects one or more image classification units based on levels of certainty acquired from the plurality of image classification units, and
the image creation unit creates one or more second images by visualizing areas contributing to the classification by the selected image classification unit(s).

3. The information processing apparatus according to claim 2,
wherein each of the plurality of image classification units acquires a level of certainty that the examination region is healthy and/or a level of certainty that the examination region has a specific disease, by inputting the first image to an inference model; and
the inference model for each of the image classification units is a model that infers whether the examination region included in the first image is healthy or has the specific disease.

4. (canceled)

5. The information processing apparatus according to claim 1, wherein the image creation unit creates the second image by adjusting, based on a level of certainty that the examination region is unhealthy, a value representing contribution to the classification by the image classification unit.

6. A method for presenting an area highly likely to be a specific area in a first image concerning an examination region of an examinee, the method comprising:
acquiring the first image;
acquiring a level of certainty that the examination region in the first image is healthy and/or a level of certainty that the examination region in the first image is unhealthy by inputting the first image to an inference model, wherein the inference model is a model obtained by using a plurality of images labeled with either healthiness or unhealthiness as ground truth data on an image by image basis;
creating a second image in which an area contributing to the level of certainty that the examination region is unhealthy is visualized; and
outputting inference results based on the acquired level of certainty and the second image.

7. A computer-readable recording medium recording a program that causes one or more computers to present an area highly likely to be a specific area in a first image concerning an examination region of an examinee, the program causing the computers to perform the processes of:
acquiring the first image;
acquiring a level of certainty that the examination region in the first image is healthy and/or a level of certainty that the examination region in the first image is unhealthy by inputting the first image to an inference model, wherein the inference model is a model obtained by using a plurality of images labeled with either healthiness or unhealthiness as ground truth data on an image by image basis;
creating a second image in which an area contributing to the level of certainty that the examination region is unhealthy is visualized; and
outputting inference results based on the acquired level of certainty and the second image.

8. An information processing apparatus for generating a learned model to be used by the information processing apparatus for presenting an area highly likely to be a specific area in an image concerning an examination region of an examinee, the information processing apparatus comprising:
a learning unit that inputs an image labeled with either healthiness or unhealthiness as ground truth data on an image by image basis to a machine learning model and causes the machine learning model to do learning; and
a model output unit that outputs a learned model caused to do learning by the learning unit.

9. The information processing apparatus according to claim 8, wherein the image includes a plurality of images labeled, as ground truth data, with either healthiness or unhealthiness regarding a plurality of diseases including diffuse diseases.

10. A method for generating a learned model to be used by an information processing apparatus for presenting an area highly likely to be a specific area in an image concerning an examination region of an examinee, the method comprising:
acquiring a plurality of images labeled with either healthiness or unhealthiness as ground truth data on an image by image basis;
causing a machine learning model to do learning using the images, wherein the machine learning model is a machine learning model that estimates whether an examination region included in the images is healthy or unhealthy; and
outputting a learned model obtained through learning done by the machine learning model.
